(19) United States
(12) Patent Application Publication
(10) Pub. No.: US 2017/0049741 A1
(43) Pub. Date: Feb. 23, 2017

(54) Formulations for Cathepsin K Inhibitors

(71) Applicants: Wayne Parent, Saint-Lazare (CA); Mahtab Afaghi, Kirkland (CA); David Breslin, Telford, PA (US); Mireille Granger, St-Laurent (CA); Lei Wang, Ambler, PA (US); Jeff Zimmerman, Sellersville, PA (US)

(72) Inventors: Wayne Parent, Saint-Lazare (CA); Mahtab Afaghi, Kirkland (CA); David Breslin, Telford, PA (US); Mireille Granger, St-Laurent (CA); Lei Wang, Ambler, PA (US); Jeff Zimmerman, Sellersville, PA (US)

(21) Appl. No.: 14/322,342

(22) Filed: Jul. 2, 2014

Related U.S. Application Data
(63) Continuation of application No. 12/527,876, filed on Aug. 20, 2009, now abandoned.
(60) Provisional application No. 60/903,493, filed on Feb. 26, 2007.

(51) Int. Cl. A61K 31/277; A61K 9/48; A61K 9/00; A61K 9/20

(52) U.S. Cl. CPC A61K 31/277; A61K 9/2018; A61K 9/2054; A61K 9/2013; A61K 9/4858; A61K 9/4866; A61K 9/0019

(57) ABSTRACT

The instant invention relates to pharmaceutical compositions containing cathespin K inhibitors. Also disclosed are processes for making said pharmaceutical compositions.

### BACKGROUND OF THE INVENTION

This invention relates to formulations of cathepsin K inhibitors.

A variety of cathepsin K inhibitors have been disclosed for the treatment of various disorders related to cathepsin K functioning, including osteoporosis, glucocorticoid induced osteoporosis, Paget's disease, abnormally increased bone turn over, tooth loss, bone fractures, rheumatoid arthritis, osteoarthritis, periprosthetic osteolysis, osteogenesis imperfecta, atherosclerosis, obesity, glaucoma, chronic obstructive pulmonary disease and cancer including metastatic bone disease, hypercalcemia of malignancy, and multiple myeloma. Representative examples of cathepsin K inhibitors include those disclosed in International Publication WO03/075836, which published on Sep. 18, 2003, to Merck & Co., Inc. & Axys Pharmaceuticals, which is hereby incorporated by reference in its entirety.

Cathepsin K inhibitors can be formulated for oral dosing as tablets, by using a direct compression, wet granulation or roller compaction method. Similarly, cathepsin K inhibitors can be formulated for oral dosing as gelatin capsules, being a liquid in a soft capsule, or dry powder or semi-solid in a hard capsule. In addition, cathepsin K inhibitors can be formulated for intravenous dosing.

### SUMMARY OF THE INVENTION

The instant invention relates to pharmaceutical compositions containing cathespin K inhibitors. Also disclosed are processes for making said pharmaceutical compositions.

### DETAILED DESCRIPTION OF THE INVENTION

A particularly effective cathepsin K inhibitor is N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide,

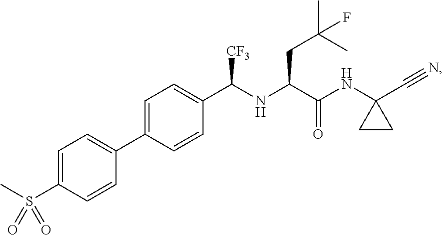 [SMILES: CC(C)(F)C[C@H](N[C@@H](c1ccc(-c2ccc(S(C)(=O)=O)cc2)cc1)C(F)(F)F)C(=O)NC1(C#N)CC1]

which can be prepared by procedures described in: International Publication WO03/075836, which published on Sep. 18, 2003, to Merck & Co., Inc. & Axys Pharmaceuticals; International Publication WO2006/017455, which published on Feb. 16, 2006, to Merck & Co., Inc.; U.S. Publication US2006-0052642, which published on Mar. 9, 2006; U.S. Publication US2005-0234128, which published on Oct. 20, 2005, to Merck & Co., Inc.; all of which are hereby incorporated by reference in their entirety.

The invention contemplates the use of any pharmaceutically acceptable fillers/compression aids, disintegrants, super-disintegrants, lubricants, binders, surfactants, film coatings, and solvents. Examples of these components are set forth below and are described in more detail in the Handbook of Pharmaceutical Excipients, Second Edition, Ed. A. Wade and P. J. Weller, 1994, The Pharmaceutical Press, London, England.

The instant invention comprises a pharmaceutical composition comprising by weight, about 0.5 to 40% by weight of a cathepsin K inhibitor, or a pharmaceutically acceptable salt thereof, and from about 60% to 99.5% by weight of excipients selected from diluents, a binder, a lubricant, and a disintegrant.

In an embodiment of the pharmaceutical composition, the excipients comprise a diluent, a binder, and a disintegrant.

In an embodiment of the invention, the cathepsin K inhibitor is N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide, or a pharmaceutically acceptable salt thereof.

In an embodiment of the invention, the diluents are selected from the group consisting of lactose anhydrous, lactose monohydrate, mannitol, microcrystalline cellulose, calcium phosphate and starch. In a class of the embodiment, the diluents are lactose monohydrate and microcrystalline cellulose.

In an embodiment of the invention, the binder is hydroxypropyl cellulose, polyvinylpyrrolidone or hydroxypropylmethylcellulose. In a class of the embodiment, the binder is hydroxypropyl cellulose.

In an embodiment of the invention, the lubricant is magnesium stearate or sodium stearyl fumerate. In a class of the embodiment, the lubricant is magnesium stearate.

In an embodiment of the invention the disintegrant is croscarmellose sodium, starch or sodium starch glycolate. In a class of the embodiment, the disintegrant is croscarmellose sodium.

The instant invention includes a process for the preparation of a tablet containing a cathepsin K inhibitor, which process comprises:

(a) forming a powder blend of the cathepsin K inhibitor with excipients,

(b) wet granulating the powder blend with hydroxypropyl cellulose to form granules,

(c) drying the granules, and

(d) compressing the dried granules in to a tablet.

In an embodiment of the process, the cathepsin K inhibitor is N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide, or a pharmaceutically acceptable salt thereof

In an embodiment of the process, the excipients comprise a diluent, a binder, and a disintegrant.

In an embodiment of the process, the diluents are selected from the group consisting of lactose anhydrous, lactose monohydrate, mannitol, microcrystalline cellulose, calcium phosphate and starch. In a class of the embodiment, the diluents are lactose monohydrate and microcrystalline cellulose.

In an embodiment of the process, the binder is hydroxypropyl cellulose, polyvinylpyrrolidone or hydroxypropylmethylcellulose. In a class of the embodiment, the binder is hydroxypropyl cellulose.

In an embodiment of the process, the lubricant is magnesium stearate or sodium stearyl fumerate. In a class of the embodiment, the lubricant is magnesium stearate.

In an embodiment of the process, the disintegrant is croscarmellose sodium, starch or sodium starch glycolate. In a class of the embodiment, the disintegrant is croscarmellose sodium.

The instant invention also includes a process for the preparation of a tablet containing a cathepsin K inhibitor, which process comprises:

(a) forming a powder blend of the cathepsin K inhibitor with excipients, using a mixer,

(b) wet granulating the powder blend with a binder to form granules,

(c) drying the granules in a fluid bed dryer,

(d) milling the dried granulate,

(e) lubricating the dried granules, and

(f) compressing the dried granules in to a tablet.

In an embodiment of the process, the cathepsin K inhibitor is N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide, or a pharmaceutically acceptable salt thereof

In an embodiment of the process, the excipients comprise a diluent, a binder, and a disintegrant.

In an embodiment of the process, the diluents are selected from the group consisting of lactose anhydrous, lactose monohydrate, mannitol, microcrystalline cellulose, calcium phosphate and starch. In a class of the embodiment, the diluents are lactose monohydrate and microcrystalline cellulose.

In an embodiment of the process, the binder is hydroxypropyl cellulose, polyvinylpyrrolidone or hydroxypropylmethylcellulose. In a class of the embodiment, the binder is hydroxypropyl cellulose.

In an embodiment of the process, the lubricant is magnesium stearate or sodium stearyl fumerate. In a class of the embodiment, the lubricant is magnesium stearate.

In an embodiment of the process, the disintegrant is croscarmellose sodium, starch or sodium starch glycolate. In a class of the embodiment, the disintegrant is croscarmellose sodium.

The instant invention also comprises a pharmaceutical composition comprising by weight, about 0.5 to 40% by weight of a cathepsin K inhibitor, or a pharmaceutically acceptable salt thereof, and from about 60% to 99.5% by weight of excipients selected from diluents and a lubricant.

In an embodiment of the invention, the cathepsin K inhibitor is N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide, or a pharmaceutically acceptable salt thereof

In an embodiment of the invention, the diluents are selected from the group consisting of lactose anhydrous, lactose monohydrate, mannitol, microcrystalline cellulose, calcium phosphate and starch. In a class of the embodiment, the diluents are lactose monohydrate and microcrystalline cellulose.

In an embodiment of the invention, the lubricant is magnesium stearate or sodium stearyl fumerate. In a class of the embodiment, the lubricant is magnesium stearate.

In an embodiment of the invention, the pharmaceutical composition also contains a binder. In a class of the embodiment, binder is hydroxypropyl cellulose, polyvinylpyrrolidone or hydroxypropylmethylcellulose. In a subclass of the embodiment, the binder is hydroxypropyl cellulose.

In an embodiment of the invention, the pharmaceutical composition consists of: 0.5 to 40% of a cathepsin K inhibitor or salt; 54% to 95.6% of a diluent or diluents; 1-2% of a lubricant. Optionally, the pharmaceutical composition can further include 3-4% dry binder. A class of the embodiment consists of 0.5 to 40% of N¹-(1-cyanocyclopropyl)-4-fluoro-N²-{(15)-2,2,2-trifluoro-1-[4′-(methylsulfonyl)-1,1′-biphenyl-4-yl]ethyl}-L-leucinamide; 27% to 47.8% of lactose (as a diluent); 27% to 47.8% of microcrystalline cellulose (as a diluent); and 1-2% of magnesium stearate.

The instant invention includes a process for the preparation of a tablet containing a cathepsin K inhibitor, which process comprises:

(a) mixing together the cathepsin K inhibitor, diluents, and a dry binder,

(b) lubricating the mixture from step (a),

(c) dry granulating the lubricated mixture,

(d) size reducing the granules,

(e) lubricating the granules, and

(f) compressing the tablets on a rotary tablet press.

In an embodiment of the process, the cathespin K inhibitor, diluent and dry binder are mixed together in a drum blender for 10 minutes. In a class of the embodiment, the drum blender is set at 46 rpm.

In an embodiment of the process, the mixture from step (a) is lubricated in a drum blender for 1 minute. In a class of the embodiment, the drum blender is set at 46 rpm.

In an embodiment of the process, the lubricated mixture from step (b) is dry granulated on a roller compactor. In a class of the embodiment, the roller compactor is set with a roll pressure of 400 MPa, a roll speed of 4.00 rpm and a screw speed of 55.5 rpm.

In an embodiment of the process, the granules from step (c) are size reduced by milling said granules through a screen and a round rasp screen. In a class of the embodiment, the screen measures 1 mm and the round rasp screen measures 1.27 mm.

In an embodiment of the process, the cathepsin K inhibitor is N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide, or a pharmaceutically acceptable salt thereof

In an embodiment of the process, the diluents are selected from the group consisting of lactose anhydrous, lactose monohydrate, mannitol, microcrystalline cellulose, calcium phosphate and starch. In a class of the embodiment, the diluents are lactose monohydrate and microcrystalline cellulose.

In an embodiment of the process, the binder is hydroxypropyl cellulose, polyvinylpyrrolidone or hydroxypropylmethylcellulose. In a class of the embodiment, the binder is hydroxypropyl cellulose.

In an embodiment of the process, the lubricant is magnesium stearate or sodium stearyl fumerate. In a class of the embodiment, the lubricant is magnesium stearate.

The instant invention also comprises an intravenous pharmaceutical composition comprising a cathepsin K inhibitor, or a pharmaceutically acceptable salt thereof, water, a modified cyclodextrin and a wetting agent.

In an embodiment of the invention, the cathepsin K inhibitor is N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide, or a pharmaceutically acceptable salt thereof

In an embodiment of the invention, the modified cyclodextrin is sulfobutyl ether-7β-cyclodextrin (Captisol®) or Hydroxypropyl beta-cyclodextrin. In a class of the embodiment, the modified cyclodextrin is sulfobutyl ether-7β-cyclodextrin.

In an embodiment of the invention, the wetting agent is polysorbate 80, polysorbate 20, poloxamer 407, poloxamer 188, Cremaphor EL or a phospholipid. In a class of the embodiment, the wetting agent is polysorbate 80.

The pharmaceutical tablet compositions of the present invention may also contain one or more additional formulation ingredients that may be selected from a wide variety of excipients known in the pharmaceutical formulation art. According to the desired properties of the tablet, any number of ingredients may be selected, alone or in combination, based upon their known uses in preparing tablet compositions. Such ingredients include, but are not limited to, diluents, binders, compression aids, disintegrants, lubricants, flavors, flavor enhancers, sweeteners, preservatives, colorants and coatings.

The term “tablet” as used herein is intended to encompass compressed pharmaceutical dosage formulations of all shapes and sizes, whether uncoated or coated. Substances which may be used for coating include hydroxypropylmethylcellulose, hydroxypropylcellulose, titanium dioxide, talc, sweeteners and colorants.

The pharmaceutical compositions of the present invention are useful in the therapeutic or prophylactic treatment of disorders associated with cathpesin K functioning. Such disorders include: osteoporosis, glucocorticoid induced osteoporosis, Paget's disease, abnormally disease, tooth loss, bone fractures, rheumatoid arthritis, osteoarthritis, periprosthetic osteolysis, osteogenesis imperfecta, atherosclerosis, obesity, glaucoma, chronic obstructive pulmonary disease and cancer including metastatic bone disease, hypercalcemia of malignancy, and multiple myeloma.

The following examples are given for the purpose of illustrating the present invention and shall not be construed as being limitations on the scope of the invention.

Ranges of Conditions for Processing:

The wet granulation processes disclosed herein can be performed in (but not limited to) high shear mixer and fluid bed processor system. Granule is then milled through a size reduction mill, lubricant is added to the granule contained in a tote, and then mixed. Granule is then compressed into tablets.

The dry granulation process can be performed in (but not limited to) a roller compactor. Granule is then milled through a size reduction mill, lubricant is added to the granule contained in a tote, and then mixed. Granule is then compressed into tablets.

### Example 1

### Preparation of 50 Mg Tablets

| | | | Weight (kg) |
|---|---|---|---|
| | % | | (Batch = |
| Component | wt./wt. | Mg/Tablet | 100,000 tablet) |
| N¹-(1-cyanocyclopropyl)-4-fluoro- | 12.5 | 50.00 | 5.0 |
| N²-{(1S)-2,2,2-trifluoro-1-[4′- | | | |
| (methylsulfonyl)-1,1′-biphenyl-4- | | | |
| yl]ethyl}-L-leucinamide | | | |
| Microcrystalline Cellulose | 40 | 160.00 | 16.0 |
| Lactose Monohydrate | 40 | 160.000 | 16.0 |
| Croscarmellose Sodium | 4 | 16.00 | 1.6 |
| Hydroxypropyl cellulose | 3 | 12.00 | 1.2 |
| Magnesium Stearate | 0.5 | 2.00 | 0.2 |
| Purified Water* | [35] | [140.00] | [14.0] |
| Total | 100 | 400.00 | 40.0 |

*removed during the during process

N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide, 4% (wt./wt.) croscarmellose sodium, and a 1:1 (wt./wt.) mixture of microcrystalline cellulose and lactose monohydrate are dry blended in a high shear mixer, and then a 3% (wt./wt.) hydroxypropyl cellulose solution is sprayed onto the mixing powders to effect granulation. The wet granulate is dried in a fluid bed dryer, the dried granulate is then milled, and finally lubricated with 0.5% (wt./wt.) magnesium stearate in a blender. Tablets were then compressed on a rotary tablet press.

### Example 2

### Preparation of 5 Mg Tablets

| Component | | | |
|---|---|---|---|
| (Batch = 100,000 tablet) | % wt./wt. | Mg/Tablet | Weight (kg) |
| N1-(1-cyanocyclopropyl)-4-fluoro- | 5 | 5 | 0.5 |
| N2-{(1S)-2,2,2-trifluoro-1-[4′- | | | |
| (methylsulfonyl)-1,1′-biphenyl-4- | | | |
| yl]ethyl}-L-leucinamide | | | |
| Microcrystalline Cellulose | 43.75 | 43.75 | 4.375 |
| Lactose Monohydrate | 43.75 | 43.75 | 4.375 |
| Croscarmellose Sodium | 4 | 4 | 0.4 |
| Hydroxypropyl cellulose | 3 | 3 | 0.3 |
| Magnesium Stearate | 0.5 | 0.5 | 0.05 |
| Purified Water* | [35] | [140.00] | [14.0] |
| Total | 100 | 100 | 100 |

*removed during the during process

N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide, 4% (wt./wt.) croscarmellose sodium, and a 1:1 (wt./wt.) mixture of microcrystalline cellulose and lactose monohydrate are dry blended in a high shear mixer, and then a 3% (wt./wt.) hydroxypropyl cellulose solution is sprayed onto the mixing powders to effect granulation. The wet granulate is dried in a fluid bed dryer, the dried granulate is then milled, and finally lubricated with 0.5% (wt./wt.) magnesium stearate in a blender. Tablets were then compressed on a rotary tablet press.

### Example 3

### Preparation of 5 Mg Tablets

| Component | | | Weight |
|---|---|---|---|
| (Batch = 100,000 tablet) | % wt./wt. | Mg/Tablet | (kg) |
| N1-(1-cyanocyclopropyl)-4-fluoro-N2- | 5 | 10 | 1 |
| {(1S)-2,2,2-trifluoro-1-[4′- | | | |
| (methylsulfonyl)-1,1′-biphenyl-4- | | | |
| yl]ethyl}-L-leucinamide | | | |
| Microcrystalline Cellulose | 43.75 | 87.5 | 8.75 |
| Lactose Monohydrate | 43.75 | 87.5 | 8.75 |
| Croscarmellose Sodium | 4 | 8 | 0.8 |
| Hydroxypropyl cellulose | 3 | 6 | 0.6 |
| Magnesium Stearate | 0.5 | 1 | 0.1 |
| Purified Water* | [35] | [140.00] | [14.0] |
| Total | 100 | 200 | 20 |

*removed during the during process

N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide, 4% (wt./wt.) croscarmellose sodium, and a 1:1 (wt./wt.) mixture of microcrystalline cellulose and lactose monohydrate are dry blended in a high shear mixer, and then a 3% (wt./wt.) hydroxypropyl cellulose solution is sprayed onto the mixing powders to effect granulation. The wet granulate is dried in a fluid bed dryer, the dried granulate is then milled, and finally lubricated with 0.5% (wt./wt.) magnesium stearate in a blender. Tablets were then compressed on a rotary tablet press.

### Example 4

### Preparation of 10 Mg Tablets

| | | | Weight (kg) |
|---|---|---|---|
| | % | | (Batch = |
| Component | wt./wt. | Mg/Tablet | 100,000 tablet) |
| N¹-(1-cyanocyclopropyl)-4-fluoro- | 10 | 10.00 | 1.00 |
| N²-{(1S)-2,2,2-trifluoro-1-[4′- | | | |
| (methylsulfonyl)-1,1′-biphenyl-4- | | | |
| yl]ethyl}-L-leucinamide | | | |
| Microcrystalline Cellulose | 42.5 | 42.50 | 4.25 |
| Lactose Monohydrate | 42.5 | 42.50 | 4.25 |
| Croscarmellose Sodium | 4 | 4.00 | 4.00 |
| Magnesium Stearate | 1 | 1.00 | 1.00 |
| Total | 100 | 100.00 | 10.00 |

N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide, and a 1:1 (wt./wt.) mixture of lactose anhydrous (type; direct tabletting), microcrystalline cellulose (type; Avicel PH102) are mixed together in a drum blender for 10 minutes at 46 rpm. The mixture is then lubricated by addition of 0.5% (wt./wt.) magnesium stearate and mixing in the same blender for 1 minute at 46 rpm. The mixture was then dry granulated on a roller compactor using the following conditions;

Roll Pressure=400 MPa

Roll Speed=4.00 rpm

Screw speed=55.5 rpm

The compacted ribbons are milled through a 1 mm screen, and then further size reduced in a cone mill equipped with a 1.27 mm round rasp screen. A final lubrication with 0.5% (wt./wt.) magnesium stearate was performed using the drum blender for 1 minute at 46 rpm. Tablets were then compressed on a rotary tablet press.

### Example 5

### Preparation of 25 Mg Soft Gelatin Capsules

| | | | Weight (kg) |
|---|---|---|---|
| | % | Mg/ | (Batch = |
| Component | wt./wt. | Capsule | 100,000 capsules) |
| N¹-(1-cyanocyclopropyl)-4-fluoro- | 2.5 | 25.00 | 2.5 |
| N²-{(1S)-2,2,2-trifluoro-1-[4′- | | | |
| (methylsulfonyl)-1,1′-biphenyl-4- | | | |
| yl]ethyl}-L-leucinamide | | | |
| PEG400 | 60 | 600.00 | 60.0 |
| Water | 10 | 100.00 | 100.0 |
| Butylated Hydroxyanisole (BHA) | 0.1 | 1.00 | 1.0 |
| Soft gelatin capsule | 27.4 | 274.00 | 27.4 |
| Total | 100 | 1000.00 | 100.0 |

N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide is dissolved in a PEG400/10% H₂O/0.1% BHA solution and then 1000 mg is filled into soft gelatin capsule. In the capsule filling process, the fill material is injected into the pocket as gelatin ribbon is molded into the capsule shape.

### Example 6

### Preparation of 10 Mg Hard Gelatin Capsules

| | | | Weight (kg) |
|---|---|---|---|
| | % | Mg/ | (Batch = |
| Component | wt./wt. | capsule | 100,000 capsule) |
| N¹-(1-cyanocyclopropyl)-4-fluoro- | 10 | 10.00 | 1.00 |
| N²-{(1S)-2,2,2-trifluoro-1-[4′- | | | |
| (methylsulfonyl)-1,1′-biphenyl-4- | | | |
| yl]ethyl}-L-leucinamide | | | |
| Microcrystalline Cellulose | 42.75 | 42.75 | 4.275 |
| Lactose Monohydrate | 42.75 | 42.75 | 4.275 |
| Croscarmellose Sodium | 4 | 4.00 | 0.4 |
| Magnesium Stearate | 0.5 | 0.5 | 0.05 |
| Total | 100 | 100.00 | 10 |
| Hard Gelatin Capsule | n/a | 40 | 4 |

N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide, and the 1:1 (wt./wt.) mixture of lactose monohydrate, microcrystalline cellulose are mixed together in a drum blender for 10 minutes at 46 rpm. The mixture is then lubricated by addition of 0.5% (wt./wt.) magnesium stearate and mixing in the same blender for 1 minute at 46 rpm. The oral gelatin capsule formulation process is performed on a dry powder filling capsule machine.

### Example 7

### Preparation of 5 Mg Hard Gelatin Capsules

| | | | Weight (kg) |
|---|---|---|---|
| | % | Mg/ | (Batch = |
| Component | wt./wt | Capsule | 100,000 capsule) |
| N¹-(1-cyanocyclopropyl)-4-fluoro- | 0.5 | 5 | 0.5 |
| N²-{(1S)-2,2,2-trifluoro-1-[4′- | | | |
| (methylsulfonyl)-1,1′-biphenyl-4- | | | |
| yl]ethyl}-L-leucinamide | | | |
| PEG4000 | 89.4 | 894 | 89.4 |
| Butylated Hydroxyanisole (BHA) | 0.1 | 1 | 0.1 |
| Water | 10 | 100.00 | 10 |
| Total | 100 | 1000.00 | 100 |
| Hard Gelatin Capsule | n/a | 75 | 7.5 |

PEG4000 is liquified at 70° C. in a non-hygroscopic environment then N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide is added with stirring to the PEG4000 until solubilized. The solution is added to the hopper* of a capsule filling machine, then hard gelatin capsules are filled with 1 g of solution. * hopper maintained at 75° C.

### Example 8

### Preparation of IV Formulation

| Component | Amount, mg/mL |
|---|---|
| N¹-(1-cyanocyclopropyl)-4-fluoro-N²- | 0.1 |
| {(1S)-2,2,2-trifluoro-1-[4′- | |
| (methylsulfonyl)-1,1′-biphenyl-4- | |
| yl]ethyl}-L-leucinamide | |
| Captisol | 350 |
| Polysorbate 80 | 0.1 |
| Water for Injection | Qs 1.00 mL |

Vehicle Preparation Procedure:

Weigh the Captisol® (0.35 g for each 1 mL of vehicle), then add the Captisol® with three times of rinse to a glass container (volumetric flask) with approximately 90% of the water. Stir the solution with a stirring bar at a speed that creates a vortex. Stir until all solid has dissolved (approximately 60 minutes). Add polysorbate 80 (0.0001 g for each 1 mL of vehicle), then Qs to the desired final volume with water. Mix well (inverting the flask by 5-6 times), and record the final pH. Filter through to the container by using Millipore GV filter unit (0.22 μm, sterile)

Formulation Preparation Procedure—0.1 mg/ml of N¹-cyanocyclopropyl)-4-fluoro-N²-{(1S)-2,2,2-trifluoro-1-[4′-(methylsulfonyl)-1,1′-biphenyl-4-yl]ethyl}-L-leucinamide) in 0.01% polysorbate 80, 35% Captisol®.

Tare the volumetric flask on the balance, add polysorbate 80 (0.1 mg for each 1 ml of vehicle). Add approximately 90% of the water weight in the formulation to a glass container (volumetric flask). Add 35% Captisol® (0.35 gram per 1 ml of water), add stirring bar to the solution, stir the solution at a speed that create a vortex, during approximately 30 minutes of stirring, invert the flask couple of times to wash off any particles on the wall of top flask. Weigh N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide (0.1 mg for each 1 ml of vehicle), then add N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide to a glass container. Sonicate for approximately 5 minutes using a bath sonicator to breakdown the large particles. Continue to stirring at 400 rpm for overnight, invert the flask if any particles were on the wall of top flask. The formulation should be clear; otherwise, continue stirring until the solution is achieved (˜24 hours). Qs to volume with water. Filter using Millipore GV filter unit (0.22 μm, sterile). Label the IV formulation and move it to 5° C. or −20° C. refrigerator immediately.

1. A pharmaceutical composition comprising by weight, about 0.5 to 40% by weight of a cathepsin K inhibitor, or a pharmaceutically acceptable salt thereof, and from about 60% to 99.5% by weight of excipients selected from diluents, a binder, a lubricant, and a disintegrant.

2. The pharmaceutical composition of claim 1 wherein the cathepsin K inhibitor is N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide, or a pharmaceutically acceptable salt thereof.

3. The pharmaceutical composition of claim 2 wherein
the diluents are selected from the group consisting of lactose anhydrous, lactose monohydrate, mannitol, microcrystalline cellulose, calcium phosphate and starch;
the binder is hydroxypropyl cellulose, polyvinylpyrrolidone or hydroxypropylmethylcellulose;
the lubricant is magnesium stearate or sodium stearyl fumerate; and
the disintegrant is croscarmellose sodium, starch or sodium starch glycolate.

4. The pharmaceutical composition of claim 3 wherein the diluents are lactose monohydrate and microcrystalline cellulose; the binder is hydroxypropyl cellulose; the lubricant is magnesium stearate; and the disintegrant is croscarmellose sodium.

5. A pharmaceutical composition comprising by weight, about 0.5 to 40% by weight of a cathepsin K inhibitor, or a pharmaceutically acceptable salt thereof, and from about 60% to 99.5% by weight of excipients selected from diluents and a lubricant.

6. The pharmaceutical composition of claim 5 wherein the cathepsin K inhibitor is N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide, or a pharmaceutically acceptable salt thereof.

7. The pharmaceutical composition of claim 6 wherein
the diluents are selected from the group consisting of lactose anhydrous, lactose monohydrate, mannitol, microcrystalline cellulose, calcium phosphate and starch; and
the lubricant is magnesium stearate or sodium stearyl fumerate.

8. The pharmaceutical composition of claim 7 wherein the diluents are lactose monohydrate and microcrystalline cellulose; and the lubricant is magnesium stearate.

9. The pharmaceutical composition of claim 5 which also contains a binder.

10. The pharmaceutical composition of claim 9 wherein the binder is hydroxypropyl cellulose, polyvinylpyrrolidone or hydroxypropylmethylcellulose.

11. The pharmaceutical composition of claim 10 wherein the binder is hydroxypropyl cellulose.

12. An intravenous pharmaceutical composition comprising N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide, or a pharmaceutically acceptable salt thereof, water, a modified cyclodextrin and a wetting agent.

13. The pharmaceutical composition of claim 1 comprising 50 mg N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide, or a pharmaceutically acceptable salt thereof.

14. The pharmaceutical composition of claim 1 comprising by weight, about 0.5 to 40% by weight of N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide, and from about 60% to 99.5% by weight of excipients selected from diluents, a binder, a lubricant, and a disintegrant.

15. A pharmaceutical composition comprising 50 mg N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide, or a pharmaceutically acceptable salt thereof, 160 mg of lactose monohydrate; 160 mg of microcrystalline cellulose; 16 mg of croscarmellose sodium; 12 mg of hydroxypropyl cellulose and 2 mg of magnesium stearate.

16. The pharmaceutical composition of claim 15 comprising 50 mg N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide, 160 mg of lactose monohydrate; 160 mg of microcrystalline cellulose; 16 mg of croscarmellose sodium; 12 mg of hydroxypropyl cellulose and 2 mg of magnesium stearate.
